(19) United States
(12) Patent Application Publication
(10) Pub. No.: US 2009/0112126 A1
(43) Pub. Date: Apr. 30, 2009

(54) STEPPED GUIDEWIRE WITH SHIM

(75) Inventors: Karl KEATING, Galway (IE); Eamon Brady, Elphin (IE); David VALE, Galway City (IE); Mars GIANOTTI, Portola Valley, CA (US); John NEILAN, Gort (IE)

Correspondence Address: Sughrue Mion-ABBOTT LABS, 2100 Pennsylvania Avenue, N.W., Washington, DC, 20037, US

(73) Assignee: SALVIAC LIMITED, Dublin 2 (IE)

(21) Appl. No.: 11/925,820

(22) Filed: Oct. 27, 2007

(51) Int. Cl. A61M 25/09

(52) U.S. Cl. 600/585; 606/108

(57) ABSTRACT

A medical guidewire (1) suitable for advancement through a vasculature comprises a core element (2), a collar element (3), a distal outer element (4), a proximal outer element (5), and a distal end cap (6). The collar element (3) is fixedly attached to the core element (2) proximally of the distal end of the guidewire (1), and extends radially outwardly from the core element (2). The collar element (3) acts as a stop to limit distal movement of a medical device over the guidewire (1).

This invention relates to a medical guidewire. In one embodiment this invention relates to a medical guidewire suitable for advancement through a vasculature to facilitate exchange of one or more medical devices over the guidewire.

### STATEMENTS OF INVENTION

According to the invention there is provided a medical guidewire comprising:-

a core element extending along at least part of the length of the guidewire; and

a collar element extending outwardly to form a stop to limit movement of a medical device relative to the guidewire.

The collar element is a particularly flexible means of achieving the stop. In particular the stiffness of the guidewire is not adversely affected by the presence of the stop.

In one embodiment of the invention the collar element extends radially outwardly. The collar element may extend outwardly from the core element. The collar element may be configured to limit distal movement of a medical device relative to the guidewire. The collar element may comprise a washer element. The collar element may comprise a shim element.

In one case the longitudinal dimension of the collar element is less than 150% of the radial dimension of the collar element. The longitudinal dimension of the collar element may be less than the radial dimension of the collar element. The longitudinal dimension of the collar element may be less than 50% of the radial dimension of the collar element. The longitudinal dimension of the collar element may be less than 30% of the radial dimension of the collar element.

In another embodiment the collar element is spaced proximally of the distal end of the guidewire. The collar element may be fixedly attached to the core element. The collar element may be formed integrally with the core element.

In another case the collar element is movable relative to the core element. The collar element may be movable along at least part of the length of the core element. The collar element may be slidable relative to the core element. The guidewire may comprise a distal stop element to limit distal movement of the collar element relative to the core element. The guidewire may comprise a proximal stop element to limit proximal movement of the collar element relative to the core element. The core element may comprise a recess for receiving at least part of the collar element.

In one embodiment the collar element extends around at least part of the circumference of the core element.

In one case the guidewire comprises at least one outer element extending along at least part of the length of the core element. The guidewire may comprise a distal outer element extending distally of the collar element. The guidewire may comprise a proximal outer element extending proximally of the collar element. The distal outer element may be formed integrally with the proximal outer element. The collar element may comprise at least one opening therethrough through which the outer element may extend. The distal outer element may be formed separately from the proximal outer element. The distal outer element may be of a different material to the proximal outer element. The outer element may comprise a coil.

In another embodiment the guidewire comprises at least one intermediate element extending along at least part of the length of the core element, the intermediate element being located between the core element and the outer element. The intermediate element may extend distally of the collar element. The intermediate element may be formed integrally with the proximal outer element. The intermediate element may comprise a coil.

### BRIEF DESCRIPTION OF THE DRAWINGS

The invention will be more clearly understood from the following description of some embodiments thereof, given by way of example only, with reference to the accompanying drawings, in which:-

FIG. 1 is a cross-sectional, side view of a distal end of a medical guidewire according to the invention;

FIGS. 2 to 7 are views similar to FIG. 1 of distal ends of other medical guidewires according to the invention;

FIG. 8 is a cross-sectional, side view illustrating assembly of a distal end of another medical guidewire according to the invention;

FIG. 9 is a cross-sectional, side view of the assembled distal end of the medical guidewire of FIG. 8;

FIGS. 10 to 12 are views similar to FIG. 1 of distal ends of other medical guidewires according to the invention;

FIG. 13 is a partially cross-sectional, side view of a distal end of another medical guidewire according to the invention;

FIGS. 14 to 18 are views similar to FIG. 1 of distal ends of other medical guidewires according to the invention;

FIG. 19 is an enlarged cross-sectional, side view of part of the medical guidewire of FIG. 18;

FIG. 20 is a view similar to FIG. 1 of a distal end of another medical guidewire according to the invention;

FIG. 21 is a partially cross-sectional, side view of a distal end of another medical guidewire according to the invention;

FIGS. 22 to 29 are views similar to FIG. 1 of distal ends of other medical guidewires according to the invention;

FIG. 30 is an end view of a collar element of a medical guidewire according to the invention prior to assembly;

FIG. 31 is an end view of the assembled collar element of FIG. 30;

FIG. 32 is an isometric view of the collar element of FIG. 30 prior to assembly;

FIGS. 33 and 34 are isometric views of collar elements of other medical guidewires according to the invention;

FIG. 35 is a view similar to FIG. 1 illustrating assembly of a distal end of another medical guidewire according to the invention;

FIG. 36 is a view similar to FIG. 1 of the assembled distal end of the medical guidewire of FIG. 35;

FIGS. 37 and 38 are views similar to FIG. 1 of distal ends of other medical guidewires according to the invention;

FIG. 39 is an isometric view of a distal end of a further medical guidewire according to the invention; and

FIGS. 40 and 41 are views similar to FIG. 1 of other medical guidewires according to the invention.

### DETAILED DESCRIPTION

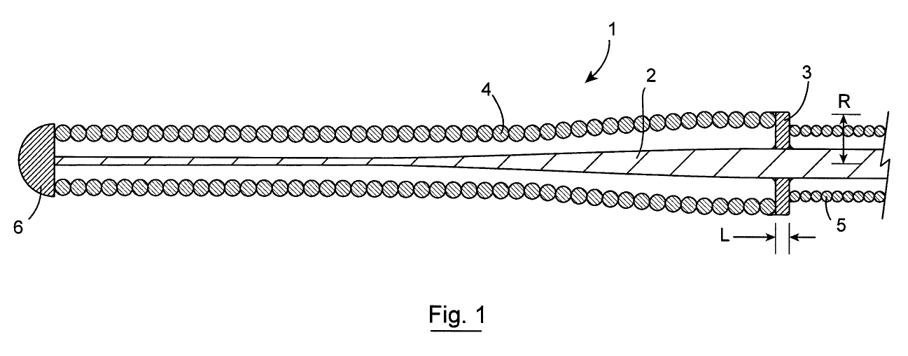

Referring to the drawings, and initially to FIG. 1 thereof, there is illustrated a medical guidewire 1 according to the invention. The guidewire 1 is suitable for advancement through a vasculature. One or more medical devices, for example a catheter or a stent, a stent delivery system, a balloon catheter, an imaging device, an embolic protection filter, or any other medical device that may be disposed over a guidewire, may be exchanged over the guidewire 1.

The guidewire 1 comprises a core element 2, a collar element 3, a distal outer element 4, a proximal outer element 5, and a distal end cap 6.

The core element 2 extends along the full length of the guidewire 1. The core element 2 tapers distally radially inwardly.

The core element 2 of the guidewires as described herein may be constructed of a unitary member or constructed of multiple pieces of material. Preferably the core element 2 is constructed of stainless steel, however other materials may be ultilized in the construction of the core element 2. Suitable materials for the core element 2 comprise nitinol, cobalt chrome, polymers, ceramics, carbon fiber, or other alloys such as those shown and described in U.S. Pat. Nos. 6,602,228 and 6,387,060 and U.S. patent application Ser. Nos. 11/019,495 and 10/409,559, the entireties of which are hereby incorporated by reference. The core element 2 may be shaped through known processes such as grinding, extruding, stamping or other known processes which will produce the core element 2 as shown and described herein.

The collar element 3 is fixedly attached to the core element 2 proximally of the distal end of the guidewire 1, and extends radially outwardly from the core element 2. The collar element 3 extends around the full circumference of the core element 2. The collar element 3 acts as a stop to limit distal movement of a medical device over the guidewire 1.

In this case the collar element 3 is provided in the form of a washer element or a shim element. The longitudinal dimension L of the collar element 3 is less than 30% of the radial dimension R of the collar element 3.

The distal outer element 4 extends along part of the length of the core element 2 distally of the collar element 3, and the proximal outer element 5 extends along part of the length of the core element 2 proximally of the collar element 3. In this case both the distal outer element 4 and the proximal outer element 5 are provided in the form of a coil. The distal outer element 4 is formed separately from the proximal outer element 5. It will be appreciated that the distal outer element 4 may be of the same material or of a different material as the proximal outer element 5. The distal outer element 4 tapers distally radially inwardly.

FIG. 1 illustrates the shim 3 (<1 mm length) laser welded to the core wire 2 to provide the step.

In use, the guidewire 1 is introduced into a vasculature and advanced through the vasculature until the guidewire 1 reaches a desired location in the vasculature. One or more medical devices may then be exchanged over the guidewire 1 to carry out a desired treatment procedure. The collar element 3 acts as a stop to limit distal movement of a medical device over the guidewire 1. In order to function as a stop as described above, the medical device disposed over the proximal outer portion 5 of the guidewire must have a guidewire lumen suitably sized to allow the device to slidably and rotatably pass over the proximal outer portion 5 but not so large as to pass over the stop 3.

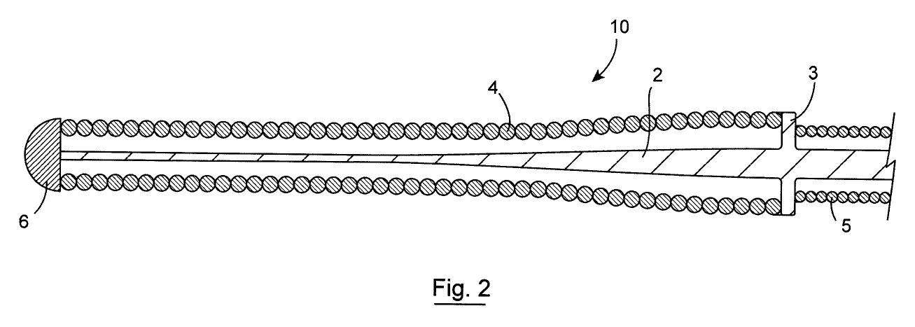

In FIG. 2 there is illustrated another medical guidewire 10 according to the invention, which is similar to the medical guidewire 1 of FIG. 1, and similar elements in FIG. 2 are assigned the same reference numerals.

In this case the collar element 3 is formed integrally with the core element 2. FIG. 2 illustrates the platinum coil 4, the step 3 ground from the SS core wire 2, the coils 4, 5 laser welded or soldered to the step 3, and the stainless steel coil 5.

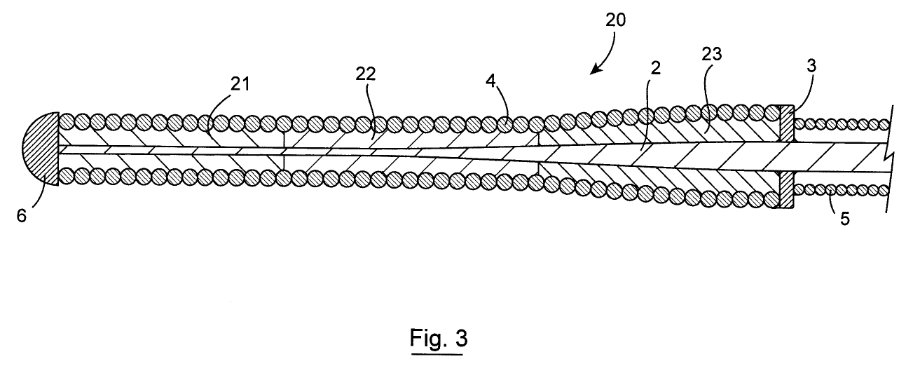

FIG. 3 illustrates another medical guidewire 20 according to the invention, which is similar to the medical guidewire 1 of FIG. 1, and similar elements in FIG. 3 are assigned the same reference numerals.

In this case the guidewire 20 comprises three intermediate elements 21, 22, 23. The intermediate elements 21, 22, 23 are located between the core element 2 and the distal outer element 4. Each of the intermediate elements 21, 22, 23 extends along part of the length of the core element 2 distally of the collar element 3.

FIG. 3 illustrates the multiple layers 21, 22, 23 of variable softness grade polymers which aid in defining distal tip flexibility. They may also be imparted with a radiopaque filler. This would allow the platinum outer coil 4 to be replaced with a SS coil or low friction sheath.

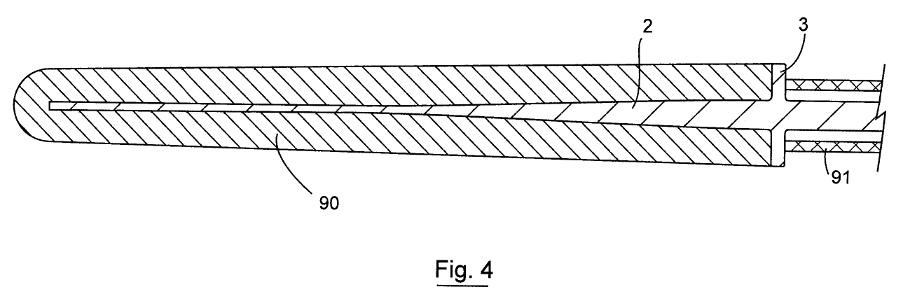

FIG. 4 illustrates a polymer 90 with a radiopaque filler, and a low friction polymer sheath 91. The polymer 90 may have low friction properties or a low friction coating may be applied.

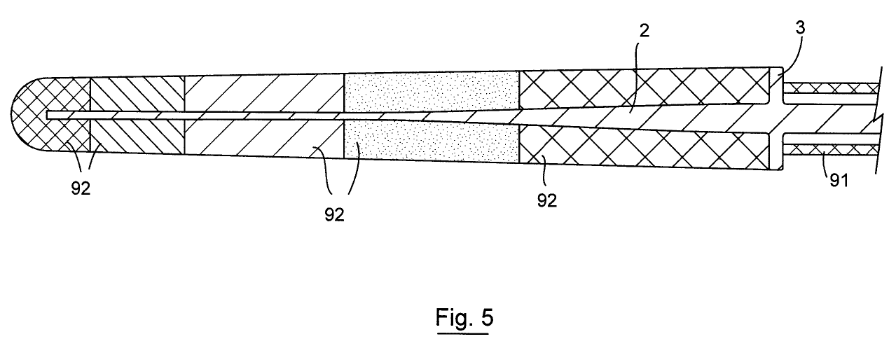

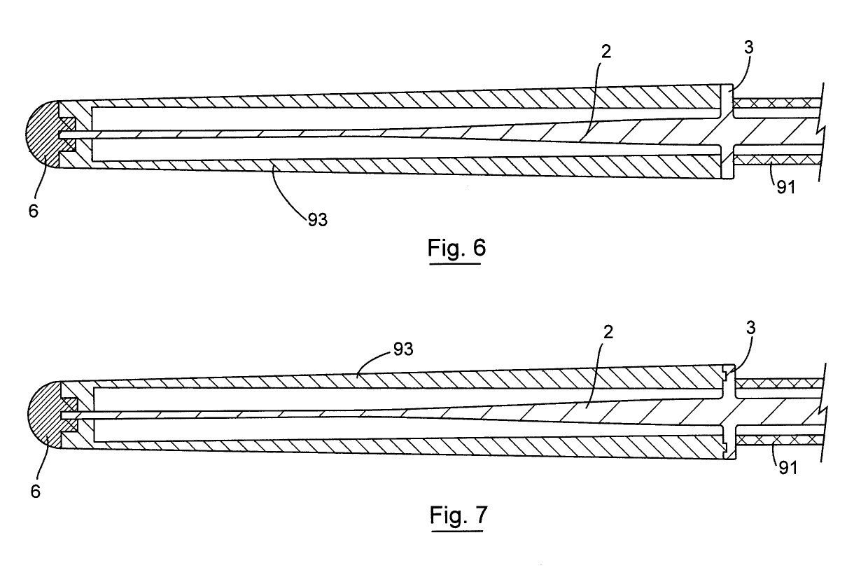

FIG. 5 illustrates layers 92 of variable stiffness polymers with a radiopaque filler. The polymer layers 92 may have low friction properties or a low friction coating may be applied.

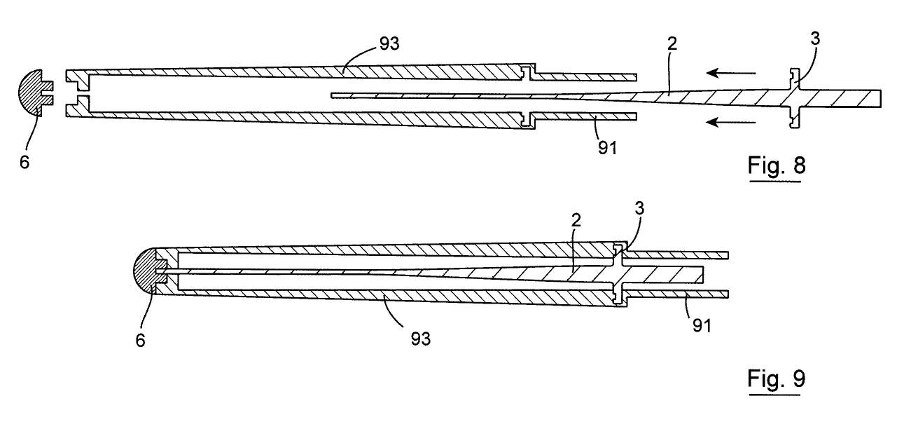

FIG. 8 illustrates a polymer sheath 93 imparted with a radiopaque filler. The sheath 93 has variable wall thickness to vary flexibility. The sheath 93 can expand over the step 3 to aid manufacturability and to anchor the sheath 93 to the guidewire. The separate nose 6 allows for a denser application of radiopaque filler to maximise radiopacity.

In FIG. 9, the sheath 93 and the nose 6 are ultrasonically/heat welded to the core wire 2 and the step 3.

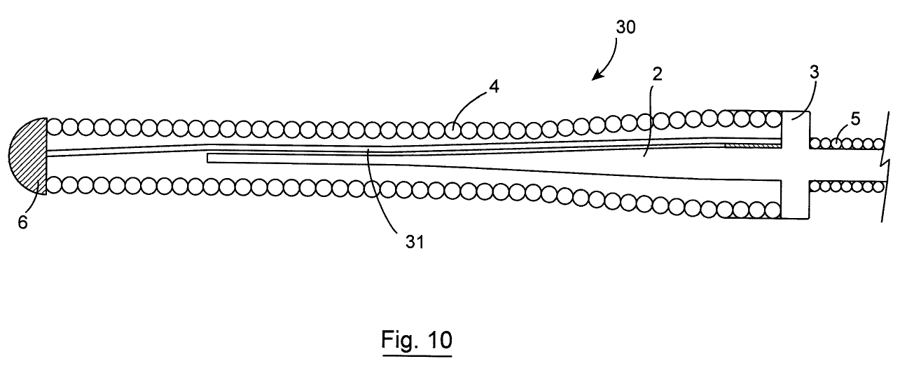

Referring to FIG. 10 there is illustrated another medical guidewire 30 according to the invention, which is similar to the medical guidewire 10 of FIG. 2, and similar elements in FIG. 10 are assigned the same reference numerals.

In this case the guidewire 30 comprises an elongate intermediate element 31 fixedly attached to the core element 2. The intermediate element 31 extends distally of the collar element 3.

The core element 2 extends along only part of the length of the guidewire 1. In particular the distal end of the core element 2 is spaced proximally of the distal end cap 6.

FIG. 10 illustrates the adhesive bond which joins the SS ribbon 31 to the NiTi core 2.

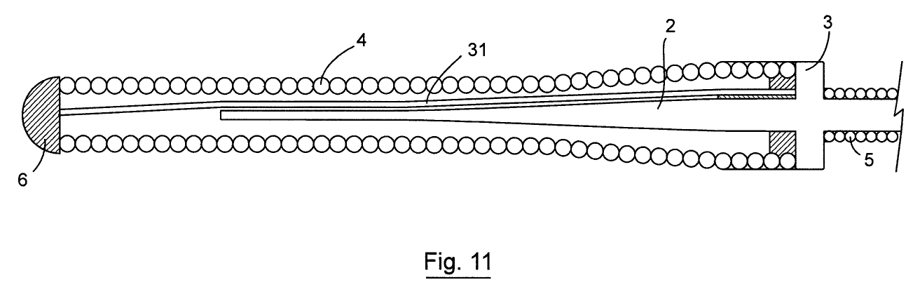

FIG. 11 illustrates the adhesive bond which joins the Pt coil 4 to the SS ribbon 31 to the NiTi core 2.

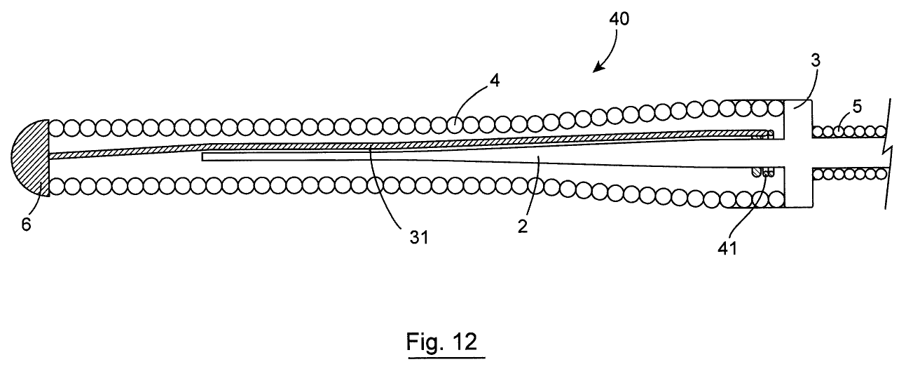

In FIG. 12 there is illustrated another medical guidewire 40 according to the invention, which is similar to the medical guidewire 30 of FIG. 10, and similar elements in FIG. 12 are assigned the same reference numerals.

In this case the intermediate element 31 comprises a coil portion 41 which extends around the core element 2. The intermediate element 31 is attached to the core element 2 at the coil portion 41.

FIG. 12 illustrates the SS ribbon 31 coiled around the core 2 and fixed in place through an adhesive bond or a laser weld or a ridge ground to the NiTi core 2.

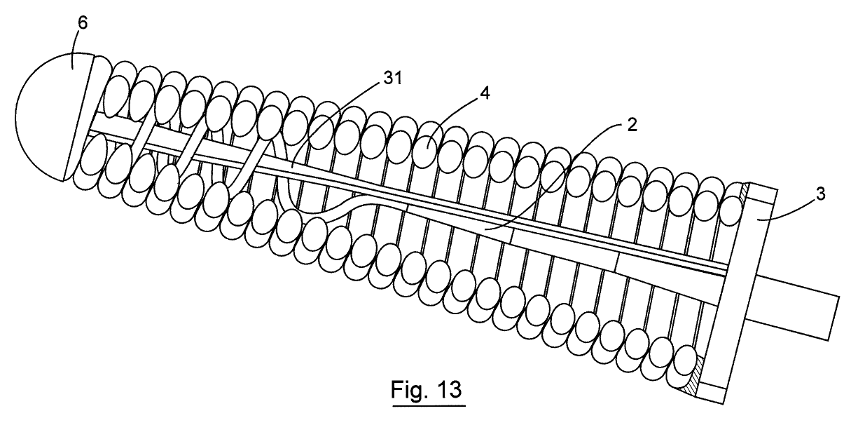

FIG. 13 illustrates the distal end of the NiTi core 2 coiled to remove resistance to bending. The coil also helps to centre the NiTi core 2 in bending. The SS ribbon 31 tapers down proximally, providing a smooth transition where the NiTi core 2 is coiled.

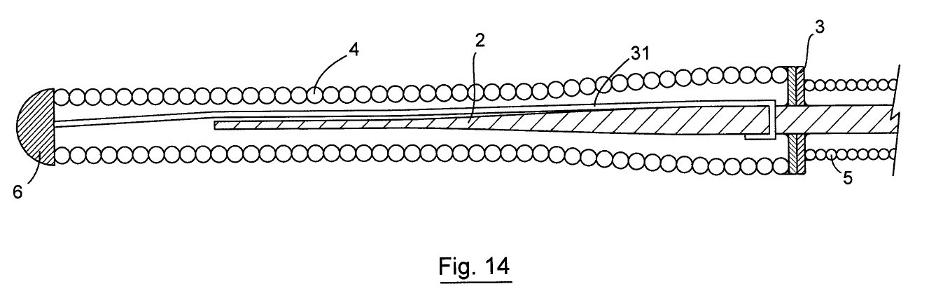

FIG. 14 illustrates the SS ribbon 31 slotted through the NiTi core 2.

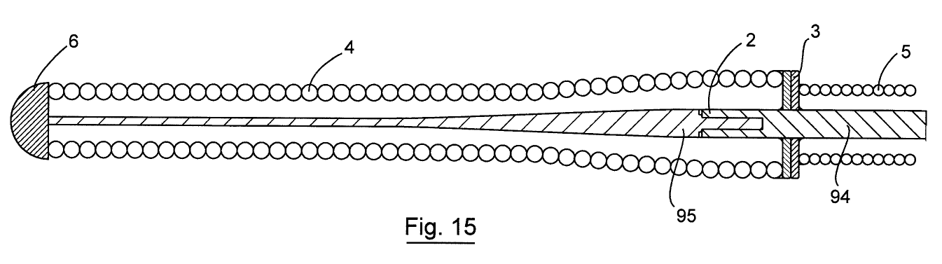

FIG. 15 illustrates the proximal NiTi core 94 heated to expand over the distal SS core 95. When cooled, this arrangement provides the attachment.

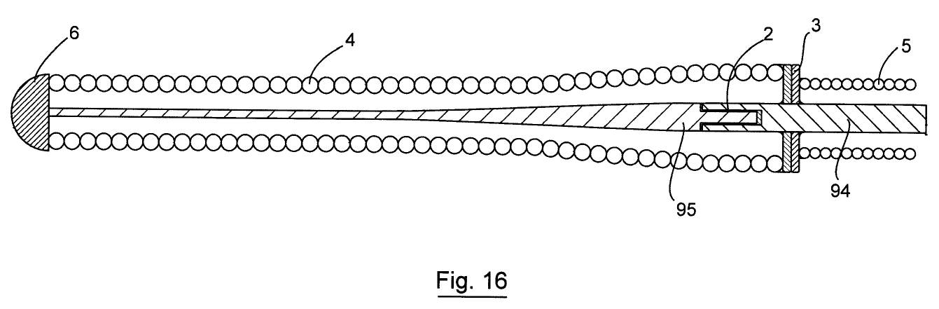

FIG. 16 illustrates the adhesive bond which joins the proximal NiTi core 94 to the distal SS core 95.

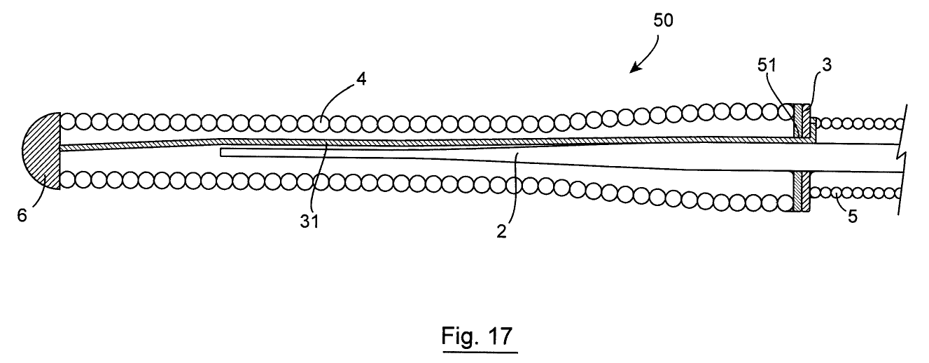

FIG. 17 illustrates another medical guidewire 50 according to the invention, which is similar to the medical guidewire 30 of FIG. 10, and similar elements in FIG. 17 are assigned the same reference numerals.

In this case the intermediate element 31 is formed integrally with the proximal outer element 5. The collar element 3 is fixedly attached to the core element 2, and has a first opening 51 extending therethrough through which the proximal outer element 5 may extend.

FIG. 17 illustrates the adjacent layer shim 3. The distal NiTi layer is laser welded to the NiTi core 2. The proximal SS layer is laser welded to the SS ribbon 31 and the SS coil 5.

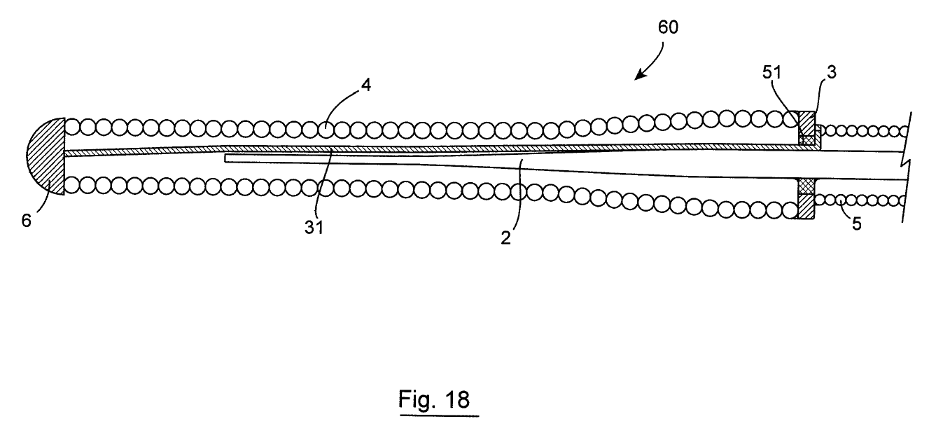

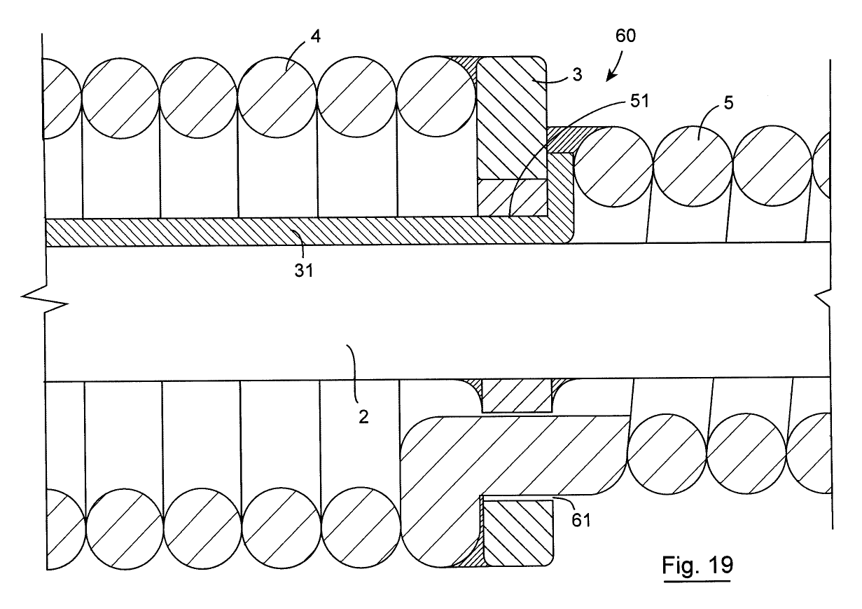

Referring to FIGS. 18 and 19 there is illustrated another medical guidewire 60 according to the invention, which is similar to the medical guidewire 50 of FIG. 17, and similar elements in FIGS. 18 and 19 are assigned the same reference numerals.

In this case the distal outer element 4 is formed integrally with the proximal outer element 5. The collar element 3 has a second opening 61 extending therethrough though which the proximal outer element 5 may extend.

FIG. 18 illustrates the platinum coil 4 laser welded to the SS layer 3. The concave layer shim 3 has an inner NiTi layer laser welded to the NiTi core 2 and an outer SS layer laser welded to the SS ribbon 31 and the SS coil 5.

FIG. 19 illustrates the distal Pt coil 5 slotted through the shim 3 to provide a single proximal/distal coil.

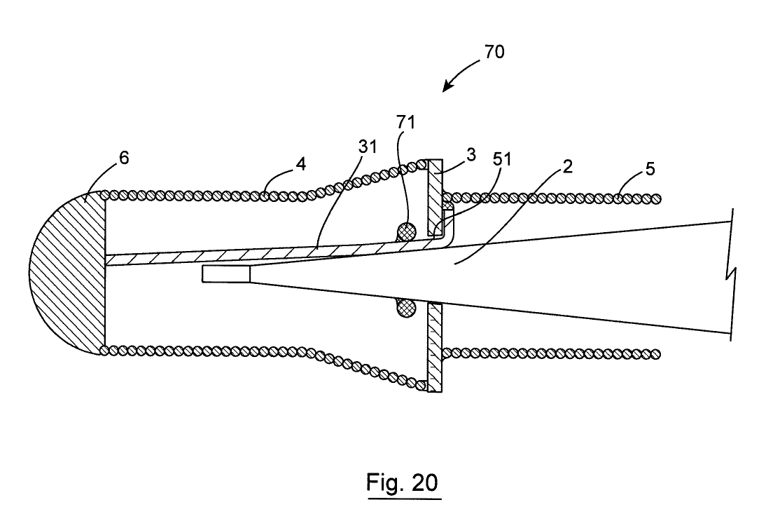

In FIG. 20 there is illustrated another medical guidewire 70 according to the invention, which is similar to the medical guidewire 50 of FIG. 17, and similar elements in FIG. 20 are assigned the same reference numerals.

In this case the collar element 3 is slidable relative to the core element 2 along part of the length of the core element 2. The guidewire 70 comprises a distal stop 71 to limit distal movement of the collar element 3 relative to the core element 2.

FIG. 20 illustrates the wire ridge 71 laser welded to the NiTi core 2. The SS ribbon 31 slots under the shim 3 and is attached thereto.

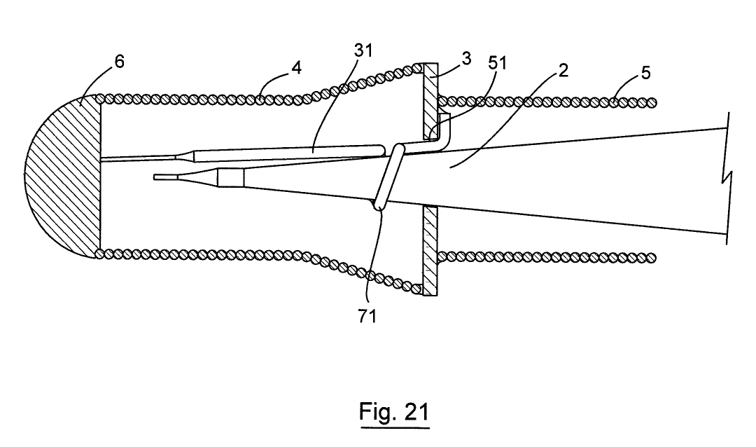

FIG. 21 illustrates the SS wire 31 coiled to provide the ridge 71. The distal end is flattened to aid steerability and shaping properties.

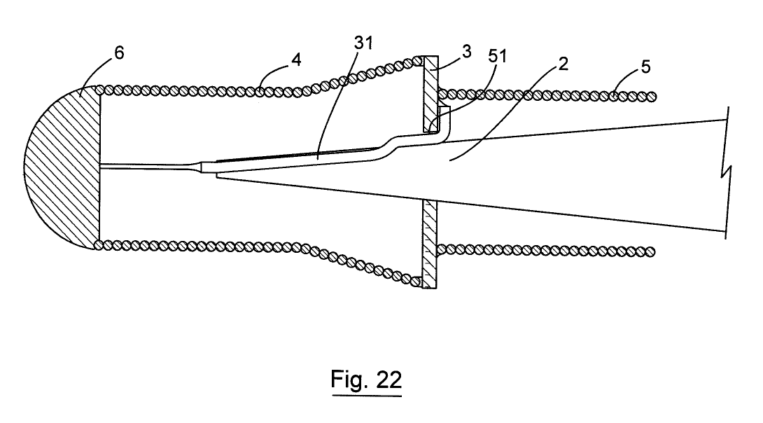

FIG. 22 illustrates the SS wire 31 slotted into a laser cut slot on the NiTi core 2. The distal end of the SS ribbon 31 is flattened to aid tip shaping and steerability properties.

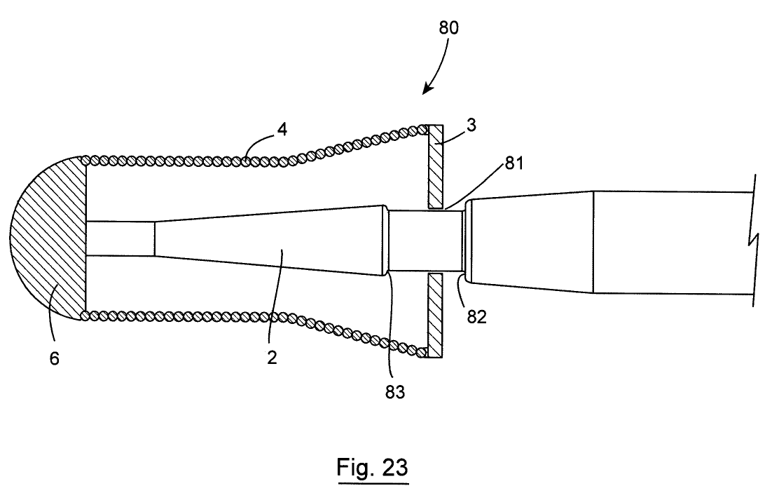

FIG. 23 illustrates another medical guidewire 80 according to the invention, which is similar to the medical guidewire 1 of FIG. 1, and similar elements in FIG. 23 are assigned the same reference numerals.

In this case the core element 2 comprises a recess 81 for receiving the collar element 3. The collar element 3 is slidable relative to the core element 2 in the recess 81. The proximal end face 82 of the recess 81 acts as a proximal stop to limit proximal movement of the collar element 3 relative to the core element 2. Similarly the distal end face 83 acts as a distal stop to limit distal movement of the collar element 3 relative to the core element 2.

No proximal outer element is provided in this case.

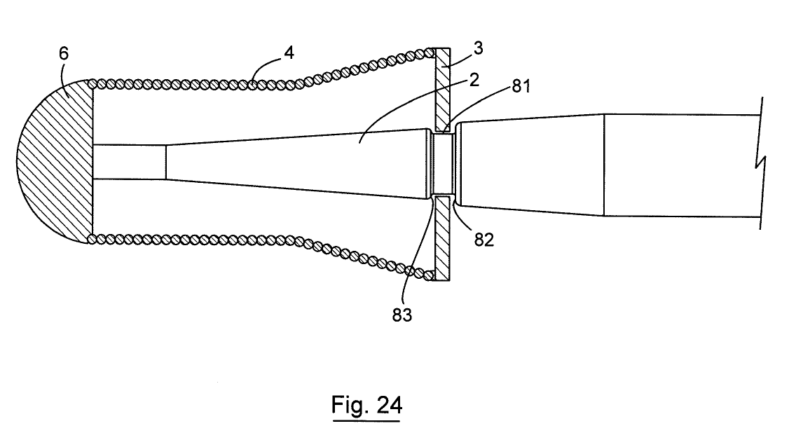

FIGS. 23 and 24 illustrate the shim 3 free to translate within the recess 81 ground in the core wire 2.

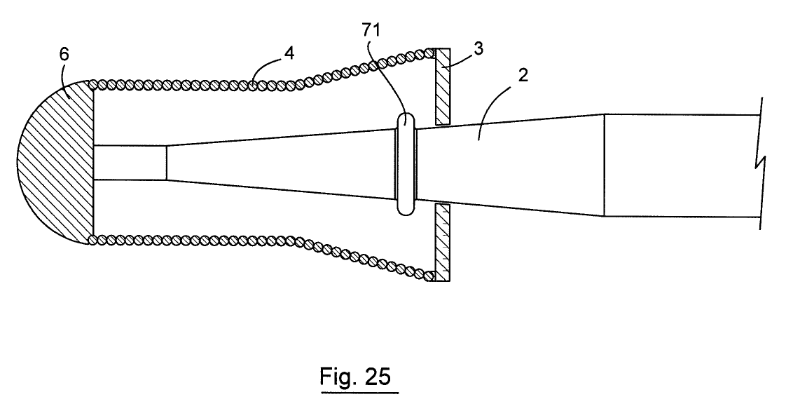

In FIG. 25 the shim 3 is free to translate between the ground ridge 71 and the tapered core 2.

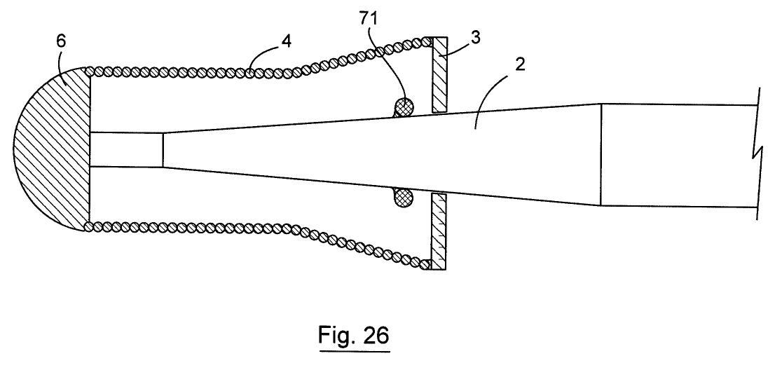

FIG. 26 illustrates the wire 71 wrapped around the core 2 and laser welded in place. The shim 3 is free to translate between the wire ridge 71 and the tapered core 2.

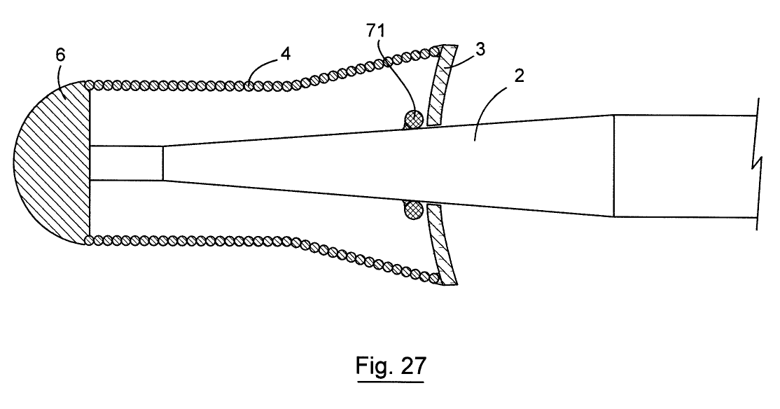

FIG. 27 illustrates the concave shim 3 acting as a spring which improves user feel when retrieving.

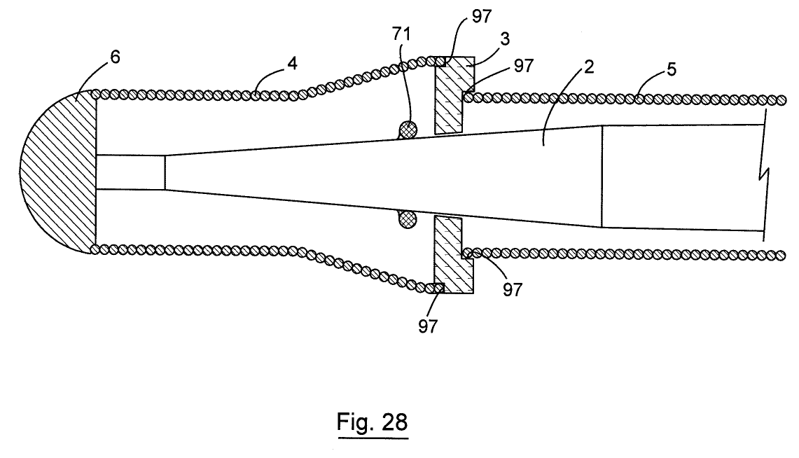

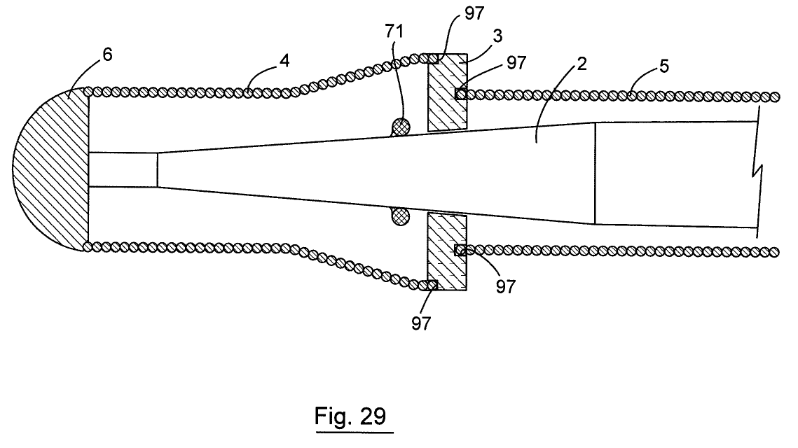

FIGS. 28 and 29 illustrate recesses 97 in the shim 3 which offer greater surface area to laserweld or solder the coils 4, 5 to the shim 3.

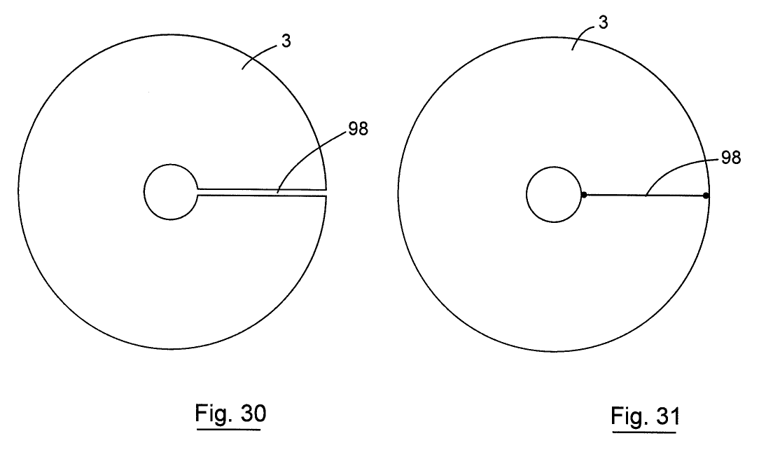

FIG. 30 illustrates the slit 98 which allows expansion of the shim 3 to fit over the ridge 71 on the core wire 2. Once in place, the shim 3 is compressed so that the internal diameter of the shim 3 is less than the outer diameter of the ridge 71. The slit 98 may be laser welded to fix in place.

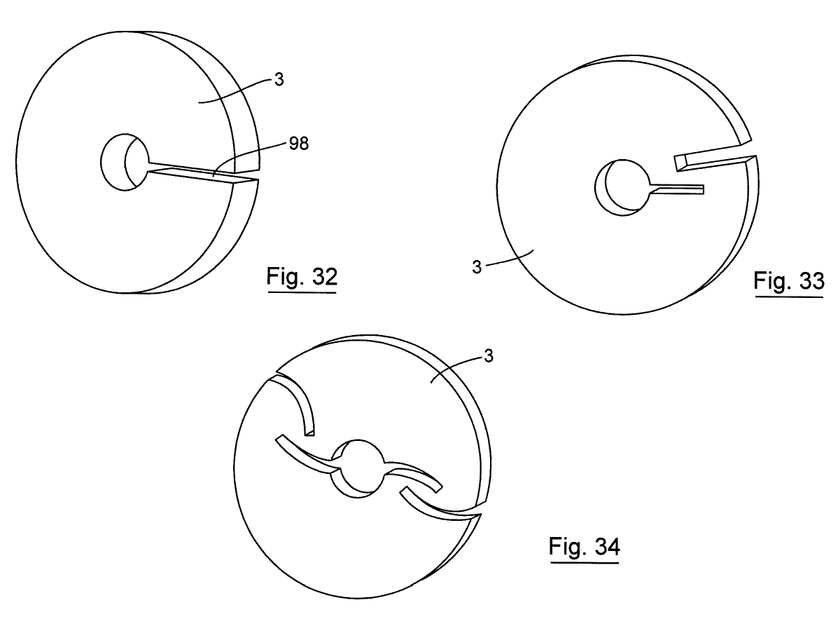

FIGS. 33 and 34 illustrate alternative expansive shim designs.

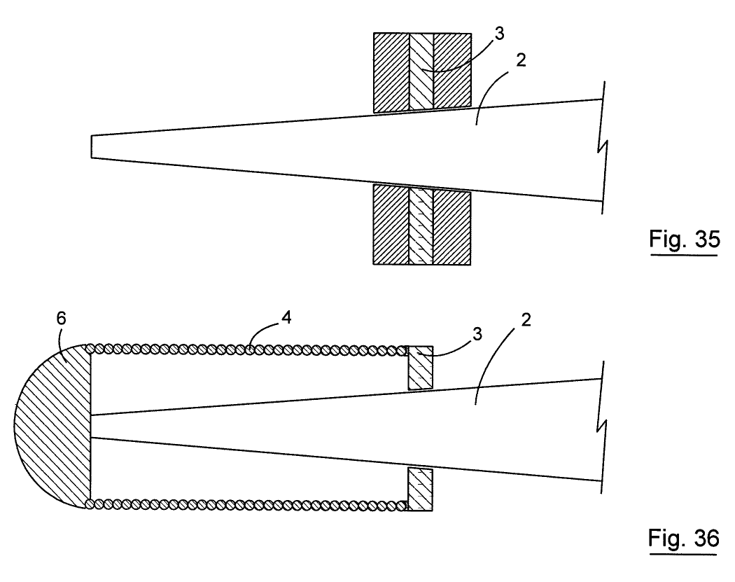

FIGS. 35 and 36 illustrate the filter abutment 3 molded to the core wire 2. The material may be solder, adhesive, polymer and/or reinforced polymer.

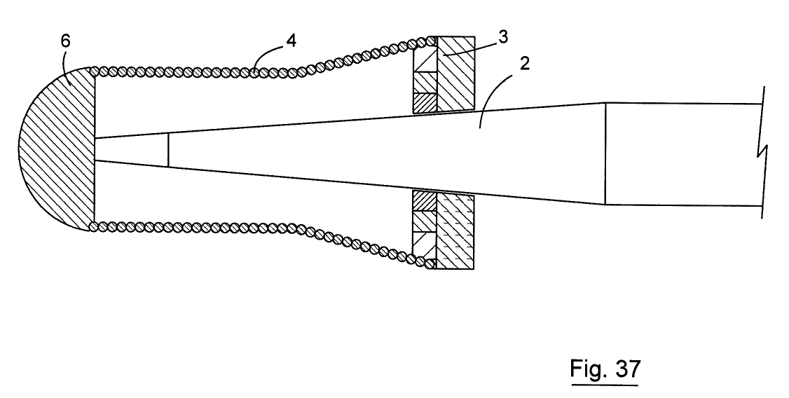

FIG. 37 illustrates the filter abutment 3 comprising several layers: solder, adhesive, polymer and/or reinforced polymer. Each layer is given a chance to solidify/cure before the next is applied. The proximal and distal faces are subsequently ground to form vertical surfaces. Post grinding, the platinum/radiopaque coil 4 is placed on top of the formed layers and bonded/joined in place.

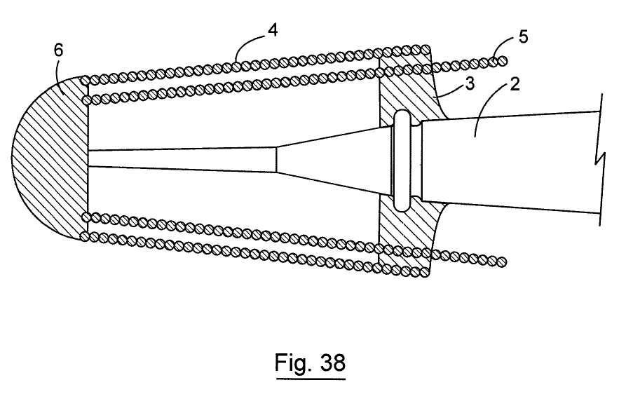

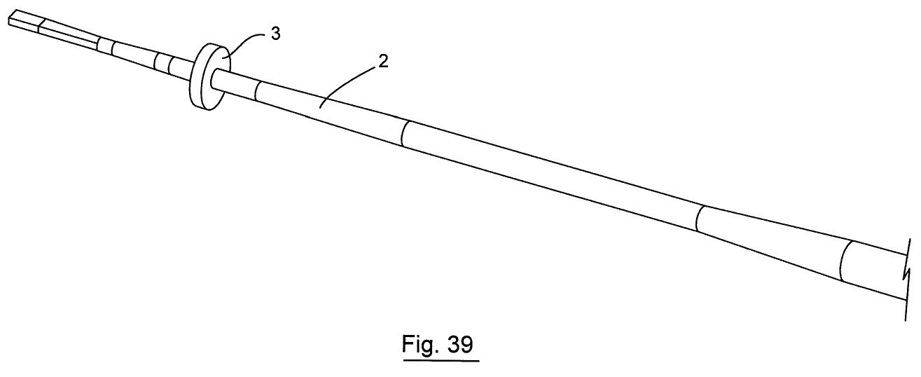

FIG. 38 illustrates the filter abutment 3 provided by a platinum/radiopaque coil bonded to the core wire 2. The dual concave and convex ridge anchors the adhesive bond to the core wire 2. Placing the convex ridge distally and the concave ridge immediately proximal provides a pool for the adhesive to settle and a wall to help restrain the adhesive from wicking distally. These features aid in keeping the step transition length to a minimum.

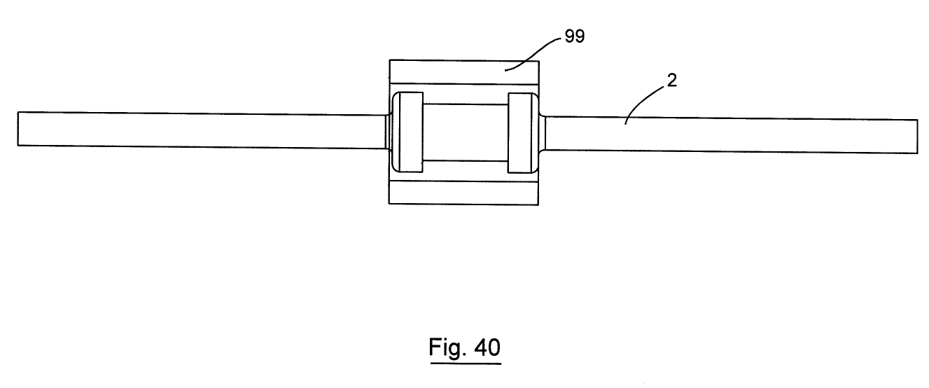

In the medical guidewire of FIG. 40 a 0.013″ [or wire original diameter] step is included when grinding and a hypotube 99 is added to achieve the 0.019″ diameter. This size of the hypotube 99 is easily attainable and can be welded/bonded/swaged or soldered in place. Joining to a 0.013″ diameter also provide a larger CSA for adhesion.

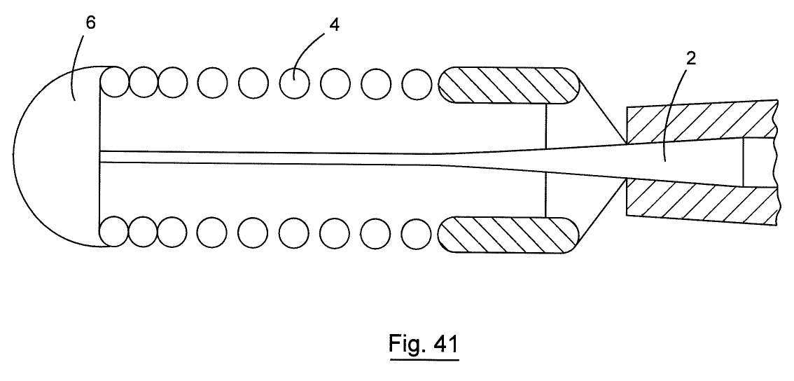

FIG. 41 illustrates a method to apply a radiopaque element to a filter delivery wire. The radiopaque element is applied to the filter delivery wire via a sputter coater. The guidewire is placed in a chamber which is at vacuum. In the case of a gold coating, Argon gas is introduced and an electric field is used to cause an electron to be removed from the Argon atoms to make the atoms ions with a positive charge. The Ar ions are then attracted to a negatively charged piece of gold foil. The Ar ions act like sand in a sandblaster, knocking gold atoms from the surface of the foil. These gold atoms settle onto the surface of the sample, producing a gold coating. The sections of the guidewire that do not require radiopaque coating will be masked in the sample chamber. Applying a radiopaque element via sputter coating allows for the incorporation of low wall thickness coating which will aid in keeping any transitions to a minimum.

The invention is not limited to the embodiments hereinbefore described, with reference to the accompanying drawings, which may be varied in construction and detail.

1. A medical guidewire comprising:-
a core element extending along at least part of the length of the guidewire; and
a collar element extending outwardly to form a stop to limit movement of a medical device relative to the guidewire.

2. A guidewire as claimed in claim 1 wherein the collar element extends radially outwardly.

3. A guidewire as claimed in claim 1 wherein the collar element extends outwardly from the core element.

4. A guidewire as claimed in claim 1 wherein the collar element is configured to limit distal movement of a medical device relative to the guidewire.

5. A guidewire as claimed in claim 1 wherein the collar element comprises a washer element.

6. A guidewire as claimed in claim 1 wherein the collar element comprises a shim element.

7. A guidewire as claimed in claim 1 wherein the longitudinal dimension of the collar element is less than 150% of the radial dimension of the collar element.

8. A guidewire as claimed in claim 7 wherein the longitudinal dimension of the collar element is less than the radial dimension of the collar element.

9. A guidewire as claimed in claim 8 wherein the longitudinal dimension of the collar element is less than 50% of the radial dimension of the collar element.

10. A guidewire as claimed in claim 9 wherein the longitudinal dimension of the collar element is less than 30% of the radial dimension of the collar element.

11. A guidewire as claimed in claim 1 wherein the collar element is spaced proximally of the distal end of the guidewire.

12. A guidewire as claimed in claim 1 wherein the collar element is fixedly attached to the core element.

13. A guidewire as claimed in claim 12 wherein the collar element is formed integrally with the core element.

14. A guidewire as claimed in claim 1 wherein the collar element is movable relative to the core element.

15. A guidewire as claimed in claim 14 wherein the collar element is movable along at least part of the length of the core element.

16. A guidewire as claimed in claim 14 wherein the collar element is slidable relative to the core element.

17. A guidewire as claimed in claim 14 wherein the guidewire comprises a distal stop element to limit distal movement of the collar element relative to the core element.

18. A guidewire as claimed in claim 14 wherein the guidewire comprises a proximal stop element to limit proximal movement of the collar element relative to the core element.

19. A guidewire as claimed in claim 1 wherein the core element comprises a recess for receiving at least part of the collar element.

20. A guidewire as claimed in claim 1 wherein the collar element extends around at least part of the circumference of the core element.

21. A guidewire as claimed in claim 1 wherein the guidewire comprises at least one outer element extending along at least part of the length of the core element.

22. A guidewire as claimed in claim 21 wherein the guidewire comprises a distal outer element extending distally of the collar element.

23. A guidewire as claimed in claim 21 wherein the guidewire comprises a proximal outer element extending proximally of the collar element.

24. A guidewire as claimed in claim 23 wherein the distal outer element is formed integrally with the proximal outer element.

25. A guidewire as claimed in claim 24 wherein the collar element comprises at least one opening therethrough through which the outer element may extend.

26. A guidewire as claimed in claim 23 wherein the distal outer element is formed separately from the proximal outer element.

27. A guidewire as claimed in claim 26 wherein the distal outer element is of a different material to the proximal outer element.

28. A guidewire as claimed in claim 21 wherein the outer element comprises a coil.

29. A guidewire as claimed in claim 21 wherein the guidewire comprises at least one intermediate element extending along at least part of the length of the core element, the intermediate element being located between the core element and the outer element.

30. A guidewire as claimed in claim 29 wherein the intermediate element extends distally of the collar element.

31. A guidewire as claimed in claim 29 wherein the intermediate element is formed integrally with the proximal outer element.

32. A guidewire as claimed in claim 29 wherein the intermediate element comprises a coil.
